(19) United States
(12) Patent Application Publication
(10) Pub. No.: US 2007/0066577 A1
(43) Pub. Date: Mar. 22, 2007

(54) BENZOXAZOLE DERIVATIVE OR ANALOGUE THEREOF FOR INHIBITING 5-LIPOXYGENASE AND PHARMACEUTICAL COMPOSITION CONTAINING SAME

(76) Inventors: Hea Young Park Choo, Seoul (KR); Hyeong-Kyu Lee, Daejeon (KR); Sei-Ryang Oh, Daejeon (KR); Kyungseop Ahn, Daejeon (KR); Gyoonhee Han, Hwaseong-si (KR); Joo Heon Kim, Daejeon (KR)

Correspondence Address: David A. Einhorn, Esq.;Anderson Kill & Olick, P.C., 1251 Avenue of the Americas, New York, NY, 10020, US

(21) Appl. No.: 11/549,280

(22) Filed: Oct. 13, 2006

Related U.S. Application Data
(63) Continuation-in-part of application No. 10/789,725, filed on Feb. 27, 2004.

(30) Foreign Application Priority Data
Apr. 3, 2003 (KR) 2003-0021055
Jul. 11, 2003 (KR) 2003-0047104

(51) Int. Cl. A61K 31/4745; A61K 31/655; A61K 31/428; A61K 31/423; C07D 498/02; C07D 491/02

(52) U.S. Cl. 514/150; 514/301; 514/302; 514/338; 514/367; 514/375; 548/152; 548/217; 534/752; 546/113; 546/114; 546/270.1; 546/271.7

(57) ABSTRACT

A novel benzoxazole derivative of formula (I) or a pharmaceutically acceptable salt thereof is effective for inhibiting 5-lipoxygenase which is useful for preventing or treating leukotriene-related diseases. The prevention also provides a pharmaceutical composition containing same and a method for preventing or treating leukotriene-related diseases.

### CROSS-REFERENCE TO RELATED APPLICATION

This application is a continuation-in-part of U.S. Ser. No. 10/789,725, filed on Feb. 27, 2004.

### FIELD OF THE INVENTION

The present invention relates to a new benzoxazole derivative or an analogue thereof.

### BACKGROUND OF THE INVENTION

Leukotriene is derived from arachidonic acid by a lipoxygenase pathway, e.g., leukotriene C₄ (LTC₄) is synthesized from arachidonic acid by the actions of 5-lipoxygenase and LTC₄ synthase. LTC₄ has long been recognized as a potent mediator of inflammation involved in diseases such as asthma, cystic fibrosis, acute/chronic bronchitis, gout, rheumatic arthritis, arthritis, allergic rhinitis, skin disorder such as psoriasis, and inflammatory bowel disease. Further, leukotriene is known to be related to various cardiopulmonary diseases including sepsis, cardiac myoischemia, cardiac anaphylaxis, cerebrovascular convulsion and ischemia, osteopososis, pain and cancer. Accordingly, a compound capable of selectively suppressing 5-lipoxygenase can be effectively used in treating the above diseases (See Curr Med. Chem.-Anti-Inflammmatory agents & Anti-allergy agents, 2003, 2, 9-18; British Journal of Pharmacology, 1989, 97(4), 1265-73; PCT WO 1999/11249 (1999 Mar. 11); Eur J. Clin. Pharmacol., 1995, 48, 155-160; Advances in prostaglandin, Thromboxane, and Leukotriene Research, 1994, 22, 113-124; European Journal of Clinical Investigation, 1995, 25, 915-919; Prostaglandins, 1987, 33(5), 663-674; Kidney International, 2002, 61, 764-776; Eur J. Med. Chem. 1997, 32, 687-707; British Journal of Pharmacology, 1988, 95, 1322-1328; British Journal of Pharmacology, 2001, 133, 1323-1329; PCT WO 2005/123130 (2005 Dec. 29); Gynecol Obset Invest., 1988, 46, 61-64; and Oncogene, 2002, 21, 5765-5772).

There have been reported various compounds suppressing 5-lipoxygenase, e.g., compounds having a hydroxyurea, hydroxamate, aryl alcohol or carboxylic acid moiety, particularly, Zileuton (Abbott Laboratories). However, these compounds including Zileuton generally suffer from the multiple problems such as liver toxicity, methemoglobin formation and poor bioavailability.

Accordingly, there has been a need to develop a drug capable of suppressing leukotriene-related diseases such as asthma and inflammation diseases by effectively inhibiting 5-lipoxygenase. The present inventors have found that benzoxazole derivatives are effective 5-lipoxygenase inhibitors.

### SUMMARY OF THE INVENTION

It is an object of the present invention to provide a new benzoxazole derivative or an analogue thereof which efficiently inhibits 5-lipoxygenase in a subject.

It is another object of the present invention to provide a pharmaceutical composition containing the benzoxazole derivative as an active ingredient for preventing or treating various leukotriene-related diseases by inhibiting 5-lipoxygenase.

In accordance with one aspect of the present invention, there is provided a benzoxazole derivative or analogue of formula (I) or a pharmaceutically acceptable salt thereof. 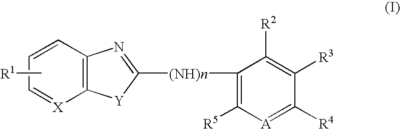
wherein
X is CH or N;
Y is S or O;
n is 0 or 1;
A is CH or N;
R¹ is H, halogen, C₁₋₆ alkyl or C₁₋₆ alkoxy;
R² is H, C₁₋₆ alkyl or halogen-substituted C₁₋₆ mercaptoalkyl;
R³ is H, halogen, C₁₋₆ alkyl, C₁₋₆ haloalkyl or C₁₋₆ alkoxy;
R⁴ is H, halogen, phenylazo, C₁₋₆ alkyl, C₁₋₆ mercaptoallcyl or C₁₋₆ alkoxy; and
R⁵ is H or C₁₋₆ alkyl.

### DETAILED DESCRIPTION OF THE INVENTION

Preferred compounds of formula (I) used in the present invention are those wherein

A is CH;

R² is H, or halogen-substituted C₁₋₆ mercaptoalkyl;

R³ is H, halogen or C₁₋₆ haloalkyl;

R⁴ is H, phenylazo, C¹⁻⁶ alkyl or C₁₋₆ mercaptoalkyl;

R⁵ is H; and

X, Y, n and R¹ are as defined in formula (I).

Most preferred compounds of formula (I) used in the present invention are those wherein X is CH, Y is O, n is 1, R¹ is C₁₋₆ alkyl, R² and R³ are H, R⁴ is C₁₋₆ alkyl, and R⁵ is H.

The representative compounds of formula (I), i.e., compounds of formula (Ia) and (Ib) can be prepared in accordance with a process shown in the following reaction scheme: 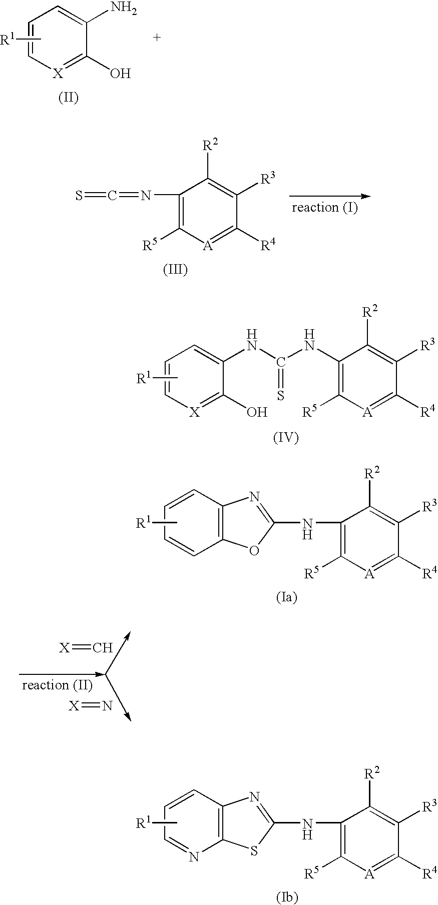
wherein R¹, R³, R⁵, R⁶, R⁷, R⁸ and R⁹ are as defined in formula (I).

As shown in the above reaction scheme, the compound of formula (II) is reacted with the compound of formula (III) in a suitable organic solvent to produce a thiourea intermediate of formula (IV) (reaction I).

The compound of formula (III) is preferably employed in an amount ranging from 1 to 1.5 equivalents, more preferably 1 to 1.2 equivalents, based on 1 equivalent of the compound of formula (II).

In accordance with a preferred embodiment of the present invention, reaction (I) is performed at or above room temperature, preferably at room temnperature, for a period ranging from 1 to 24 hours, preferably over 12 hours. It is also possible to carry out the reaction for over 24 hours.

Preferred organic solvents that may be used in the present invention include methanol, ethanol, ether and the like, and methanol is most preferred.

The thiourea intermediate of formula (IV) is obtained as a precipitate, and the end point of the reaction (I) may be identified by thin-layer chromatography.

The resulting thiourea intermediate of formula (IV) is then cyclized by reacting with an acid to synthesize the compound of formula (Ia) (R³=H) (reaction II). The acid is added in an amount sufficient to dissolve the thiourea intermediate.

The reaction (II) is performed at a temperature ranging from a room temperature to a reflux temperature, preferably, the reflux temperature, for a period ranging from 1 to 24 hours, preferably over 12 hours. It is also possible to carry out the reaction for over 24 hours.

Exemplary acids that may be used in the present invention include trifluoroacetic acid, phosphoric acid, sulfuric acid, hydrochloric acid and nitric acid; while trifluoroacetic acid and phosphoric acid are more preferred; with trifluoroacetic acid being most preferred.

After completion of the reaction, the acid is removed using any of the conventional methods to obtain a desired product. For example, when trifluoroacetic acid is used, rotaty evaporation may be used.

Furthermore, the compounds of formulae (Ia) and (Ib), wherein each R³ is C₁₋₆ alkyl, can be prepared ftom the corresponding compounds wherein each R³ is H, by a conventional substitution reaction process (reactions III and IV).

The inventive compound of formula (I) can be administered to a patient in the form of a pharmaceutical composition comprising the compound or a pharmaceutically acceptable salt thereof.

Exemplary pharmaceutically acceptable salts that may be used in the present invention include therapeutically active, non-toxic acid-addition salts of the compound of formula (I).

These salts can be prepared by treating the compound of formula (I) with a suitable acid, for example, hydrochloric acid, hydrobromic acid, sulfuric acid, nitric acid, phosphoric acid, acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid and p-toluenesulfonic acid.

Due to its 5-lipoxygenase inhibiting activity, the compound of formula (I) is an effective agent for preventing or treating a leukotriene-related disease in human, e.g., asthma, pertussis, psoriasis, rheumatic arthritis, arthritis, inflammatory bowel disease, cystic fibrosis, acute/chronic bronchitis, gout, sepsis, cardiac myoischemia, cardiac anaphylaxis, ischemia, allergic rhinitis, osteopososis, pain and cancer.

When used for the above purposes, said compound may be administered via the oral, parenteral or topical route. The compound may be administered as is but is preferably administered in the form of a composition which is formulated with a pharmaceutically acceptable carrier and optional excipients, flavors, adjuvants, etc. in accordance with good pharmaceutical practice.

The composition may be in the form of a solid, semi-solid or liquid dosage form: such as tablet, capsule, pill, powder, suppository, solution, elixir, syrup, suspension, cream, lozenge, paste and spray. As those skilled in the art would recognize, depending on the chosen route of administration, the composition form of said 5-lipoxygenase inhibitor is determined. In general, it is preferred to use a unit dosage form of the inventive inhibitor in order to achieve an easy and accurate administration of the active compound. In general, the therapeutically effective compound of formula (I) is present in such a dosage form at a concentration level ranging from about 0.5% to about 90% by weight of the total composition, i.e., in an amount sufficient to provide the desired unit dose.

The 5-lipoxygenase inhibitor compound of formula (I) may be administered in single or multiple doses. The particular route of administration and the dosage regimen will be determined by the attending physician in keeping with the condition of the individual to be treated and said individual's response to the treatment. For oral administration, doses of from about 10 to about 1000 mg/kg per day in single or multiple doses may be sufficient. For parenteral administration, doses of from about 5 to 800 mg/kg per day may be used in single or multiple doses. In some instances, dosage levels below the lower limit of the aforesaid range may be more than adequate, while in other cases still larger doses may be employed without causing any harmful or deleterious side effects, provided that such higher dose levels are first divided into several smaller doses that are to be administered throughout the day.

For oral administration, tablets containing various excipients such as sodium citrate, calcium carbonate and dicalcium phosphate may be employed along with various disintegrants such as starch and preferably potato or tapioca starch, alginic acid and certain complex silicates, together with binding agents such as polyvinylpyrrolidone, sucrose, gelatin and acacia. Additionally, lubricating agents such as magnesium sterate, sodium lauryl sulfate and talc are often used for tabletting. Solid compositions of a similar type may also be employed as fillers in soft and hard-filled gelatin capsules; preferred materials in this connection include lactose or milk sugar as well as high molecular weight polyethylene glycols. When aqueous suspensions and/or elixirs are desired for oral administration, the active ingredient may be combined with various sweetening or flavoring agents, colorants or dyes, and, if so desired, emulsifying and/or suspending agents as well, together with such diluents as water, ethanol, propylene glycol, glycerin and various like combinations thereof.

The present invention also provides a pharmaceutical composition in a unit dosage form for the inhibition of 5-lipoxygenase activity in a patient in need of such treatment, comprising a compound of formula (I) and one or more nontoxic pharmaceutically acceptable carriers, adjuvants or vehicles. The amount of the active ingredient that may be combined with such materials to produce a single dosage form will vary depending upon various factors, as indicated above.

A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol.

Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions.

Suppositories for rectal administration of the compound of formula (I) can be prepared by mixing the drug with a suitable nonirritating excipient such as cocoa butter and polyethylene glycols, which are solid at ordinary temperatures but liquid at body temperature and which therefore melt in the rectum and release the drug.

Additionally, it is also possible to administer the aforesaid compounds topically and this may be preferably done by way of cream, salve, jelly, paste, ointment and the like, in accordance with the standard pharmaceutical practice.

The following Examples are intended to further illustrate the present invention without limiting its scope.

### EXAMPLE 1

### Preparation of 2-[N-(2-Ethylphenyl)]aminopyridinothiazole

2-Hydroxy-3-aminopyridine (0.92 mmol) and phenyl isothiocyanate (0.92 mmol) in methanol (50 ml) was stirred at room temperature for a day. The precipitate was filtered and washed with methanol to obtain N-(2-hydroxypyridino)-N′-(2-ethylphenyl) thiourea as a yellow powder. N-(2-hydroxypyridino)-N′-(4-ethylphenyl) thiourea (0.41 mmol) was then treated with trifluoroacetic acid (5 ml), refluxed for a day, trifluoroacetic acid was removed by rotary evaporation, and the crude product was purified by column chromatography (ethyl acetate:hexane=3:1 v/v) to obtain the title compound as a pale brown powder.

The compounds obtained in Example 1 and characteristic properties thereof are shown in Table 1.

TABLE 1

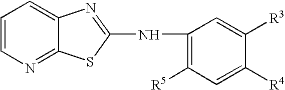

| Ex. | R³ | R⁴ | R⁵ | Data |
|---|---|---|---|---|
| 1 | H | H | C₂H₅ | 2-[N-(2-Ethylphenyl)] aminopyridinothiazol (1) |
| | | | | mp: 178˜179□ |
| | | | | ¹HNMR (Acetone-d₆, 400 MHz) δ1.209(t, J=7.6 |
| | | | | Hz, 3H), 2.774(q, J=7.6 Hz, 2H), 7.220-7.277(m, |
| | | | | 1H), 7.273˜7.320(m, 2H), 7.344-7.368(m, 1H), |
| | | | | 7.719(dd, J=1.2 and 8.0 Hz, 1H), 7.819(dd, J= |
| | | | | 1.2 and 7.6 Hz, 1H), 8.182(m, 1H), 9.009(brs, |
| | | | | NH). FABHRMS (m/z): 256.0908 (M⁺+1, |
| | | | | requires C₁₄H₁₄N₃S: |
| | | | | 256.0909) |

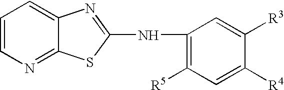

| Ex. | R³ | R⁴ | R⁵ | Data |
|---|---|---|---|---|
| 1 | H | H | C₂H₅ | 2-[N-(2-Ethylphenyl)]aminopyridinothiazol (1) |
| | | | | mp: 178˜179□ |
| | | | | ¹HNMR (Acetone-d₆, 400 MHz) δ1.209(t, J=7.6 |
| | | | | Hz, 3H), 2.774(q, J=7.6 Hz, 2H), 7.220˜7.277(m, |
| | | | | 1H), 7.273˜7.320(m, 2H), 7.344˜7.368(m, 1H), |
| | | | | 7.719(dd, J=1.2 and 8.0 Hz, 1H), 7.819(dd, J= |
| | | | | 1.2 and 7.6 Hz, 1H), 8.182(m, 1H), 9.009(brs, |
| | | | | NH). |
| | | | | FABHRMS (m/z): 256.0908 (M⁺+1, requires |
| | | | | C₁₄H₁₄N₃S: 256.0909) |

### EXAMPLE 2

### Preparation of 8-Methoxy-2-(N-phenyl)aminobenzoxazole

2-Aminophenol (0.92 mmol) and phenyl isothiocyanate (0.92 mmol) in methanol (50 ml) was stirred at room temperature for a day. The precipitate was filtered and washed with ether (7 ml) to obtain N-(2-hydroxy-5-methoxy-phenyl)-N′-phenyl thiourea as a white powder. A solution of N-(2-hydroxy-5-methoxy-phenyl)-N′-phenyl thiourea (1 mmol) in CH₃CN (3 ml) was added to a heterogeneuous solution of potassium superoxide (5 mmol) in CH₃CN (2 ml) at 20° C. under dry nitrogen atmosphere, The mixture was stirred well for 12 hr at 20° C., poured into cold water and extracted with dichloromethane. The resultant was dried over anhydrous magnesium sulfate, filtered, and concentrated under reduced pressure to obtain the title compound as a yellow powder.

### EXAMPLES 3 TO 19

Various Benzoxazole compounds were obtained by the procedure of Example 2.

The compounds obtained in Examples 2 to 19 and characteristic properties thereof are shown in Table 2.

TABLE 2

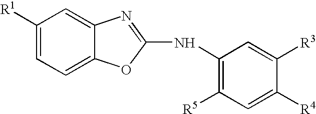

| Ex. | R¹ | R³ | R⁴ | R⁵ | Data |
|---|---|---|---|---|---|
| 2 | CH₃O | H | H | H | 8-methoxy-2-(N-phenyl) |
| | | | | | aminobenzoxazole (2) |
| | | | | | mp: 213.7˜214.5□, |
| | | | | | yield: 75% |
| | | | | | ¹H NMR (Acetone-d₆, |
| | | | | | 400 MHz) δ3.794(s, 3H), |
| | | | | | 6.663(dd, J=2.4 and 8.4 |
| | | | | | Hz, 1H), 6.992(d, J=2.4 |
| | | | | | Hz, 1H), 7.006˜7.048(m, |
| | | | | | 1H), 7.231(d, J=8.4 Hz, |
| | | | | | 1H), 7.323˜7.373(m, |
| | | | | | 2H), 7.802˜7.836(m, |
| | | | | | 2H), 9.429(brs, NH). |
| | | | | | FABHRMS (m/z): |
| | | | | | 241.0980 (M⁺+1, |
| | | | | | C₁₄H₁₃N₂O₂ |
| | | | | | requires 241.0977). |
| 3 | CH₃O | H | C₂H₅ | H | 8-Methoxy-2-[N-(4- |
| | | | | | ethylphenyl)] |
| | | | | | aminobenzoxazole (3) |
| | | | | | mp: 122.4˜125.6□, |
| | | | | | yield: 55% |
| | | | | | ¹H NMR (Acetone-d₆, |
| | | | | | 400 MHz) δ 1.215(t, J= |
| | | | | | 7.6 Hz, 3H), 2.623(q, J= |
| | | | | | 7.6 Hz, 2H), 3.821(s, |
| | | | | | 3H), 6.675(dd, J=8.4 |
| | | | | | and 2.4 Hz, 1H), 6.999 |
| | | | | | (d, J=2.4 Hz, 1H), |
| | | | | | 7.214˜7.255(m, 3H), |
| | | | | | 7.732˜7.767(m, 2H). |
| | | | | | FABHRMS (m/z): |
| | | | | | 269.1290 (M⁺+ |
| | | | | | 1, C₁₆H₁₇N₂O₂ |
| | | | | | requires: 269.1294). |
| 4 | CH₃O | Cl | Cl | H | 8-methoxy-2-[N-(3,4- |
| | | | | | dichlorophenyl)] |
| | | | | | aminobenzoxazole (4) |
| | | | | | mp: 185.2˜190.1□, |
| | | | | | yield: 56% |
| | | | | | ¹H NMR (Acetone-d₆, |
| | | | | | 400 MHz) δ 3.840(s, |
| | | | | | 3H), 6.738(dd, J=8.8 and |
| | | | | | 2.4 Hz, 1H), 7.08(d, J= |
| | | | | | 2.4 Hz, 1H), 7.298(d, J= |
| | | | | | 8.8 Hz, 1H), 7.556(d, J= |
| | | | | | 8.8 Hz, 1H), 7.709(dd, |
| | | | | | m, J=8.8 and 2.4 Hz, |
| | | | | | 1H), 8.299(d, J=2.4 Hz, |
| | | | | | 1H). |
| | | | | | FABHRMS (m/z): |
| | | | | | 309.0198 (M⁺+ |
| | | | | | 1, C₁₄H₁₁Cl₂N₂O₂ |
| | | | | | requires 309.0198). |
| 5 | CH₃O | H | H | C₂H₅ | 8-Methoxy-2-[N-(2- |
| | | | | | ethylphenyl)] |
| | | | | | aminobenzoxazole (5) |
| | | | | | mp: 208.7˜210.5□, |
| | | | | | yield: 90% |
| | | | | | ¹H NMR (Acetone-d₆, |
| | | | | | 400 MHz), δ1.106(t, J= |
| | | | | | 7.6 Hz, 3H), 2.680(q, J= |
| | | | | | 7.6 Hz, 2H), 3.673(s, |
| | | | | | 3H), 6.527(dd, 2.8 and |
| | | | | | 8.4 Hz, 1H), 6.978˜ |
| | | | | | 7.019(m, 1H), 7.086˜ |
| | | | | | 7.108(m, 1H), 7.135(˜ |
| | | | | | 7.019(m, 2H), 7.918˜ |
| | | | | | 7.939(d, J=8.4 Hz, |
| | | | | | 1H), 8.359(brs, NH). |
| 6 | CH₃O | H | CH(CH₃)₂ | H | 8-Methoxy-2-[N-(4- |
| | | | | | isopropylphenyl)] |
| | | | | | amminobenzoxazole (6) |
| | | | | | mp: 129.6˜132.5□, |
| | | | | | yield: 63% |
| | | | | | ¹H NMR (Acetone-d₆, |
| | | | | | 400 MHz) δ 1.103(s, |
| | | | | | 3H), 1.120(s, 3H), |
| | | | | | 2.918(m, 1H), 3.689(s, |
| | | | | | 3H), 6.543(dd, J= |
| | | | | | 8.4 and 2.8 Hz, 1H), |
| | | | | | 6.865(d, J=2.8 Hz, |
| | | | | | 1H), 7.110(d, J=8.4 |
| | | | | | Hz, 1H), 7.110˜ |
| | | | | | 7.139(m, 2H), 7.603˜ |
| | | | | | 7.638(m, 2H). |
| | | | | | FABHRMS (m/z): |
| | | | | | 283.1447 (M⁺+ |
| | | | | | 1, C₁₇H₁₉N₂O₂ |
| | | | | | requires 283.1445). |
| 7 | CH₃O | H | SCH₃ | H | 8-Methoxy-2-[N-(4- |
| | | | | | methylthiophenyl)] |
| | | | | | aminobenzoxazole (7) |
| | | | | | mp: 176.4˜178.8□, |
| | | | | | yield: 74% |
| | | | | | ¹H NMR (Acetone-d₆, |
| | | | | | 400 MHz) δ 2.485(s, |
| | | | | | 3H), 3.825(s, 3H), |
| | | | | | 6.692(dd, J=8.8 |
| | | | | | and 2.8 Hz, 1H), |
| | | | | | 7.013(s, J=2.8 Hz, |
| | | | | | 1H), 7.257(d, J= |
| | | | | | 8.8 Hz, 1H), 7.333˜ |
| | | | | | 7.355(m, 2H), 7.801˜ |
| | | | | | 7.837(m, 2H), |
| | | | | | FABHRMS (m/z): |
| | | | | | 287.0854 (M⁺+ |
| | | | | | 1, C₁₅H₁₅N₂O₂S |
| | | | | | requires 287.0848). |
| 8 | CH₃O | Br | H | H | 8-Methoxy-2-[N-(3- |
| | | | | | bromophenyl)] |
| | | | | | aminobenzoxazole (8) |
| | | | | | mp: 211.5˜213.1□ |
| | | | | | ¹H NMR (Acetone-d₆, |
| | | | | | 400 MHz) δ 3.839(s, |
| | | | | | 3H), 6.724(dd, J=8.8 |
| | | | | | and 2.4 Hz, 1H), |
| | | | | | 7.068(d, J=2.4 |
| | | | | | Hz, 1H), 7.228(m, 1H), |
| | | | | | 7.285(d, J=8.8 Hz, |
| | | | | | 1H), 7.325(t, J= |
| | | | | | 8 Hz, 1H), 7.718(m, |
| | | | | | 1H). |
| | | | | | FABHRMS (m/z): |
| | | | | | 319.0082 (M⁺+ |
| | | | | | 1, C₁₄H₁₂BrN₂O₂ |
| | | | | | requires 319.0086) |
| 9 | CH₃ | H | N═N—Ph | H | 8-Methyl-2-[N-(4- |
| | | | | | phenylazophenyl)] |
| | | | | | aminobenzoxazole (9) |
| | | | | | mp: 255˜258□, |
| | | | | | yield: 76% |
| | | | | | ¹H NMR (Acetone-d₆, |
| | | | | | 400 MHz) δ2.423(s, 3H), |
| | | | | | 7.354(d, J=8.0 Hz, |
| | | | | | 1H), 7.301(d, J=8.0 |
| | | | | | Hz, 1H), 7.335(s, 1H), |
| | | | | | 7.497˜7.540(m, 1H), |
| | | | | | 7.557˜7.603(m, 2H), |
| | | | | | 7.903˜7.933(m, 2H), |
| | | | | | 7.999˜8.074(m, 2H), |
| | | | | | 8.079˜8.102(m, 2H), |
| | | | | | 9.881(s, NH). |
| | | | | | FABHRMS (m/z): |
| | | | | | 329.1398(M⁺+ |
| | | | | | 1, C₂₀H₁₇N₄O |
| | | | | | requires 329.1402) |
| 10 | CH₃ | H | C₂H₅ | H | 8-Methyl-2-[N-(4- |
| | | | | | ethylphenyl)] |
| | | | | | aminobenzoxazole (10) |
| | | | | | mp: 184˜186.2□, |
| | | | | | yield: 79% |
| | | | | | ¹H NMR (Acetone-d₆, |
| | | | | | 400 MHz) δ1.364(t, J= |
| | | | | | 6.8 Hz, 3H), 2.383(s, |
| | | | | | 3H), 4.041(q, J=6.8 |
| | | | | | Hz, 2H), 6.895˜ |
| | | | | | 6.918(m, 1H), 6.933˜ |
| | | | | | 6.965(m, 2H), 7.199˜ |
| | | | | | 7.219(m, 2H), 7.722˜ |
| | | | | | 7.762(m, 2H), 9.210(s, |
| | | | | | NH). |
| | | | | | FABHRMS (m/z): |
| | | | | | 253.1345 (M⁺+ |
| | | | | | 1, C₁₆H₁₇N₂O |
| | | | | | requires 253.1341) |
| 11 | CH₃ | Cl | Cl | H | 8-Methyl-2-[N-(3,4- |
| | | | | | dichlorophenyl)] |
| | | | | | aminobenzoxazole (11) |
| | | | | | mp: 177.1˜178.3□, |
| | | | | | yield: 56% |
| | | | | | ¹H NMR (Acetone-d₆, |
| | | | | | 400 MHz) δ2.409(s, 3H), |
| | | | | | 6.99(dd, J=1.6 and 8.0 |
| | | | | | Hz, 1H), 7.280(d, J= |
| | | | | | 8.0 Hz, 1H), 7.314(d, J= |
| | | | | | 1.6 Hz, 1H), 7.557(d, J= |
| | | | | | 8.8 Hz, 1H), 7.731(dd, |
| | | | | | J=8.8 and 2.4 Hz, 1H), |
| | | | | | 8.287(d, J=2.4 Hz, |
| | | | | | 1H), 9.750(s, NH). |
| | | | | | FABHRMS (m/z): |
| | | | | | 293.0244 (M⁺+ |
| | | | | | 1, C₁₄H₁₁Cl₂N₂O |
| | | | | | requires 293.0248) |
| 12 | CH₃ | H | OC₂H₅ | H | 8-Methyl-2-[N-(4- |
| | | | | | ethoxyphenyl)] |
| | | | | | aminobenzoxazole (12) |
| | | | | | mp: 177.1˜178.3□, |
| | | | | | yield: 62% |
| | | | | | ¹H NMR (Acetone-d₆, |
| | | | | | 400 MHz), δ1.364(t, J= |
| | | | | | 6.8 Hz, 3H), 2.383(s, |
| | | | | | 3H), 4.041(q, J= |
| | | | | | 6.8 Hz, 2H), 6.895˜ |
| | | | | | 6.918(m, 1H), 6.933˜ |
| | | | | | 6.965(m, 2H), 7.199˜ |
| | | | | | 7.219(m, 2H), 7.722˜ |
| | | | | | 7.762(m, 2H), 9.210(s, |
| | | | | | NH). |
| | | | | | FABHRMS (m/z): |
| | | | | | 269.1292 (M⁺+ |
| | | | | | 1, C₁₆H₁₇N₂O₂ |
| | | | | | requires 269.1290) |
| 13 | CH₃ | H | H | C₂H₅ | 8-Methyl-2-[N-(2- |
| | | | | | ethylphenyl)] |
| | | | | | aminobenzoxazole (13) |
| | | | | | mp: 127.3˜128.4□, |
| | | | | | yield: 92% |
| | | | | | ¹H NMR (Acetone-d₆, |
| | | | | | 400 MHz) δ 1.237(t, |
| | | | | | J=7.6 Hz, 3H), |
| | | | | | 3.275(s, 3H), 2.781(q, |
| | | | | | J=7.6 Hz, 2H), 6.907(d, |
| | | | | | J=8.0 Hz), 7.240˜ |
| | | | | | 7.300(m, 2H), 8.075(d, |
| | | | | | J=8.0 Hz, 1H), 8.499(s, |
| | | | | | NH). |
| | | | | | FABHRMS (m/z): |
| | | | | | 253.1346(M⁺+ |
| | | | | | 1, C₁₆H₁₇N₂O |
| | | | | | requires 253.1341). |
| 14 | CH₃ | H | CH(CH₃)₂ | H | 8-Methyl-2-[N-(4- |
| | | | | | isopropylphenyl)] |
| | | | | | aminobenzoxazole (14) |
| | | | | | mp: 183.0˜186.9□, |
| | | | | | yield: 56% |
| | | | | | ¹H NMR (Acetone-d₆, |
| | | | | | 400 MHz) δ1.234(s, 3H), |
| | | | | | 1.251(s, 3H), 2.390(s, |
| | | | | | 3H), 2.905(m, 1H), |
| | | | | | 6.913˜6.935(m, 1H), |
| | | | | | 7.217˜7.271(m, 4H), |
| | | | | | 7.749˜7.771(m, 2H). |
| | | | | | FABHRMS (m/z): |
| | | | | | 267.1498(M⁺+ |
| | | | | | 1, C₁₁H₁₉N₂O |
| | | | | | requires 267.1497). |
| 15 | CH₃ | H | OCH₃ | H | 8-Methyl-2-[N-(4- |
| | | | | | methoxyphenyl)] |
| | | | | | aminobenzoxazole (15) |
| | | | | | mp: 180.5˜181.9□, |
| | | | | | yield: 74% |
| | | | | | ¹H NMR (Acetone-d₆, |
| | | | | | 400 MHz) δ2.394(s, 3H), |
| | | | | | 3.808(s, 3H), 6.890˜ |
| | | | | | 6.920(m, 2H), 6.947˜ |
| | | | | | 6.970(m, 2H), 7.200˜ |
| | | | | | 7.220(m, 1H), 7.741˜ |
| | | | | | 7.765(m, 2H). |
| | | | | | FABHRMS (m/z): |
| | | | | | 255.1136 (M⁺+ |
| | | | | | 1, C₁₅H₁₅N₂O₂ |
| | | | | | requires 255.1134). |
| 16 | CH₃ | H | SCH₃ | H | 8-Methyl-2-[N-(4- |
| | | | | | methylthiophenyl)] |
| | | | | | aminobenzoxazole (16) |
| | | | | | mp: 1187.0˜190.8□, |
| | | | | | yield: 69% |
| | | | | | ¹H NMR (Acetone-d₆, |
| | | | | | 400 MHz) δ2.394(s, 3H), |
| | | | | | 2.485(s, 3H), 6.941(d, |
| | | | | | J=8.0 Hz, 1H), 7.240(d, |
| | | | | | J=8.0 Hz, 2H), 7.326˜ |
| | | | | | 7.355(m, 2H), 7.807˜ |
| | | | | | 7.843(m, 2H), 9.447(s, |
| | | | | | NH). |
| | | | | | FABHRMS (m/z): |
| | | | | | 271.0905 (M⁺+ |
| | | | | | 1, C₁₅H₁₅N₂OS |
| | | | | | requires 271.0905). |
| 17 | CH₃ | Br | H | H | 8-methyl-2-[N-(3- |
| | | | | | bromohenyl)] |
| | | | | | aminobenzoxazole (17) |
| | | | | | mp: 188.4˜189.8□, |
| | | | | | yield: 94% |
| | | | | | ¹H NMR (Acetone-d₆, |
| | | | | | 400 MHz) δ2.406(s, 3H), |
| | | | | | 6.962˜6.987(m, 1H), |
| | | | | | 7.213˜7.345(m, 4H), |
| | | | | | 7.726˜7.754(m, 1H), |
| | | | | | 8.270(s, 1H), 9.633(s, |
| | | | | | NH). |
| | | | | | FABHRMS (m/z): |
| | | | | | 303.0138 (M⁺+ |
| | | | | | 1, C₁₄H₁₂BrN₂O |
| | | | | | requires: 303.0133). |
| 18 | CH₃ | OCH₃ | H | H | 8-Methyl-2-[N-(3- |
| | | | | | methoxyphenyl)] |
| | | | | | aminobenzoxazole (18) |
| | | | | | mp: 164.8˜166.4□, |
| | | | | | yield: 72% |
| | | | | | ¹H NMR (Acetone-d₆, |
| | | | | | 400 MHz) δ2.396(s, 3H), |
| | | | | | 6.623˜6.652(m, 1H), |
| | | | | | 6.931˜6.57(m, 1H), |
| | | | | | 7.233˜7.266(m, 3H), |
| | | | | | 7.328˜7.356(m, 1H), |
| | | | | | 7.621(m, 1H). |
| | | | | | FABHRMS (m/z): |
| | | | | | 255.1128 (M⁺+ |
| | | | | | 1, C₁₅H₁₅N₂O₂ |
| | | | | | requires 255.1134). |
| 19 | H | H | N═N—Ph | H | 2-[N-(4-Phenyl- |
| | | | | | azophenyl)] |
| | | | | | aminobenzoxazole (19) |
| | | | | | mp: 209-210.5° C. |
| | | | | | IR (KBr): 3384, 2922, |
| | | | | | 2852, 1664, 1460, 1019, |
| | | | | | cm⁻¹. |
| | | | | | ¹H NMR (acetone-d₆, |
| | | | | | 400 MHz): δ 9.97(s, |
| | | | | | NH), 8.12-8.10(d, |
| | | | | | J=8 Hz, 2H), 8.05˜ |
| | | | | | 8.03(d, J=8 Hz, 2H), |
| | | | | | 7.94-7.92(d, J= |
| | | | | | 8 Hz, 2H), 7.61- |
| | | | | | 7.57(t, J=12 Hz, |
| | | | | | 2H), 7.55-7.51(t, |
| | | | | | J=12 Hz, 2H), 7.46- |
| | | | | | 7.444(d, J=8 Hz, 1H), |
| | | | | | 7.30-7.26(t, J=12 |
| | | | | | Hz, 1H), 7.21-7.17(t, |
| | | | | | J=12 Hz, 1H). |
| | | | | | FAB/MS (m/z): 315 |
| | | | | | (M⁺+1). |

### EXAMPLE 20

### Preparation of 2-phenyl benzothiazole

1 g of 2-chlorotrytyl chloride resin (1.66 mmol/g, 1 eq) was allowed to swell in methylene chloride for 3-5 min, 1.162 mmol of N,N-diisopropylethylamine (0.202 ml, 0.7 eq) and 1.66 mmol of aminothiophenol (0.178 ml, 1 eq) were added thereto, and the mixture was gently stirred for 3 hr at room temperature. Then, the resin was filtered and washed with a mixture of methylene chloride, methanol and N,N-diisopropylethylamine (85:10:5 v/v/v) to obtain 2-chlorotrityl resin loaded with 0.332 mmol/g of 2-aminobenzenethiol. The loaded 2-chlorotrityl resin 200 mg was suspended in 5-6 ml of N,N-dimethylformamide, 0.996 mmol of benzoyl chloride (0.116 ml, 3 eq) and 0.996 mmol of N,N-diisopropylethylamine (0.173 ml, 3 eq) were added thereto, and the mixture was shaken for 3 hr at room temperature. Then, the resin was filtered and washed with 5×10 ml N,N-dimethylformamide and 5×10 ml methylene chloride. The resin still remaining on the filter was then treated with 20 ml portions of 65% trifluoroacetic acid/methylene chloride and 5% triethylsilane/methylene chloride. The obtained filtrates were concentrated in a vacuum to obtain an oily residue, which was dissolved in 10 ml of N,N-dimethylformamide/methanol (9:1 v/v) containing 0.2 mmol of dithiothreitol (0.031 g). After 3 hr of standing at room temperature, the mixture was extracted with ether, washed with water, dried and concentrated in a vacuum to obtain the title compound as a white powder.

### EXAMPLES 21 TO 25

Various benzothiazole compounds were obtained by the procedure of Example 20.

The compounds obtained in Examples 20 to 25 and characteristic properties thereof are shown in Table 3.

TABLE 3

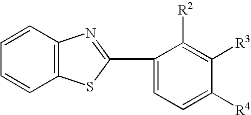

| Ex. | R² | R³ | R⁴ | Data |
|---|---|---|---|---|
| 20 | H | H | NO₂ | 2-(4-nitrophenyl) |
| | | | | benzothiazole (20) |
| | | | | mp: 232-233.7° C. |
| | | | | ¹H NMR (acetone-d₆, |
| | | | | 400 MHz): δ 8.42(s, 4H), |
| | | | | 8.18-8.16(d, J=8 Hz, |
| | | | | 1H), 8.14-8.12(d, J=8 |
| | | | | Hz, 1H), 7.63-7.59(t, |
| | | | | J=12 Hz,, 1H), 7.55- |
| | | | | 7.51 (t, J=12 Hz, 1H). |
| | | | | FAB/MS (m/z): |
| | | | | 242 (M⁺+1). |
| 21 | SCHF₂ | H | H | 2-(2-difluoromethyl- |
| | | | | thiophenyl) |
| | | | | benzothiazole (21) |
| | | | | mp: 188-190° C. |
| | | | | ¹H NMR (Acetone-d6) δ |
| | | | | 8.07(1H, s) 8.00(1H, dd, J= |
| | | | | 0.8 and 8.0 Hz), 7.94˜ |
| | | | | 7.90(2H, m) 7.87(1H, J= |
| | | | | 0.8 and 8.0 Hz) 7.45(1H, |
| | | | | dt, J=1.2 and 8.0 Hz). |
| 22 | H | H | O(CH₂)₃CH₃ | 2-(4-n-butoxymethyl- |
| | | | | phenyl)benzothiazole (22) |
| | | | | ¹H NMR (Acetone-d6) δ |
| | | | | 8.03˜8.07(3H, m) 7.50(1H, |
| | | | | dt, J=1.2 and 8.4 Hz) |
| | | | | 7.39(1H, dt, J=1.2 and 8.4 |
| | | | | Hz) 7.07˜7.11(2H, m) |
| | | | | 4.10(2H, t, J=6.4 Hz) |
| | | | | 1.71˜1.81(2H, m) |
| | | | | 1.47˜1.54(2H, m) |
| | | | | 0.97(3H, t, J=7.6 Hz) |
| | | | | FAB/MS (m/z): |
| | | | | 284 (M⁺+1). |
| 23 | H | H | CH₃ | 2-(4-methylphenyl) |
| | | | | benzothiazole (23) |
| | | | | mp: 80˜82° C. |
| | | | | ¹H NMR (Acetone-d6) δ |
| | | | | 8.07(1H, dd, J=0.8 |
| | | | | and 8.4 Hz) 8.00˜8.03(3H, |
| | | | | m) 7.54(1H, dt, J=1.2 and |
| | | | | 8.0 Hz) 7.44(1H, dt, J= |
| | | | | 1.2 and 8.0 Hz) 2.43(3H, |
| | | | | s). |
| | | | | FAB/MS (m/z): |
| | | | | 248 (M⁺+Na). |
| 24 | H | CH₂Cl | H | 2-(3-chloromethylphenyl) |
| | | | | benzothiazole (24) |
| | | | | ¹H NMR (Acetone-d6) δ |
| | | | | 7.94(1H, br d J=1.2 and |
| | | | | 7.6 Hz) 7.92˜7.88(2H, m) |
| | | | | 7.41(1H, dt, J=1.2 and |
| | | | | 8.0 Hz) 7.31(1H, dt, J= |
| | | | | 1.2 and 8.0 Hz) 7.29˜ |
| | | | | 7.24(2H, m), 2.30(2H, s). |
| | | | | FAB.MS (m/z): |
| | | | | 260(M⁺+1). |

| | | |
|---|---|---|
| 25 | 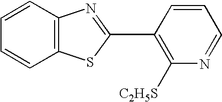 | 2-(2-ethylthiopyridin-3-yl) benzothiazole (25) ¹H NMR (Acetone-d6) δ 8.59(1H, dd, J=1.2 and 8.0 Hz) 8.26(1H, dd, J=1.2 and 8.0 Hz) 8.06˜8.15(2H, m) 7.58(1H, dt, J=1.2 and 8.4 Hz) 7.50(1H, dt, J=1.2 and 8.4) 7.29(1H, d, J=8.0 Hz). FAB/MS (m/z): 273(M⁺+1). |

### TEST EXAMPLE 1

### In vitro Measurement of 5-lipoxygenase Inhibition Activity

Bone marrow cells extracted from male BALB/cJ mice were cultured in a 1:1 (v/v) mixture of an enriched medium (RPMI 1640 medium containing penicillin 100 units/ml, streptomycin 100 mg/ml, gentamycin 10 mg/ml, 2 mM L-glutamate, 0.1 mM nonessential amino acids and 10% fetal bovine serum) and WEHI-3 cell conditioned medium as a source of interlukin-3 for 10 weeks. 3 weeks after the initiation of culture, over 90% of cells were confirmed as mast cells originated from bone marrow.

The obtained cells were suspended in the enriched medium to a concentration of 1×10⁶ cells/ml. Then, a test compound prepared by dissolving each of the compounds of Examples 1 to 25 in dimethylsulfoxide (DMSO) was added therero to a concentration of 2.5 μg/ml, and the cells were cultured in a CO₂ incubator at 37° C. for 30 min. 100 ng/ml of stem cell factor (SCF) was added to the culture medium, and then, the culture was centrifuged at 120×g for 20 min at 4° C. Then, the supernatant was separated and the amount of free LTC₄ was determined using an LTC₄ enzyme immunoassay kit (Cayman Chemical, Ann Arbor, Mich., U.S.A.). The stem cell factor (SCF) was recombinantly expressed by the baculovirus/insect cell expression system. After 20-min stimulation, the supermatants were isolated for further analysis by enzyme immunoassay.

IC₅₀ values, i.e., the concentrations of each test compound reducing the enzyme activity by 50% as compared the non-treated control, are shown in Table 4.

TABLE 4

| Example | IC₅₀(μM) |
|---|---|
| 1 | 6.82 |
| 2 | 7.88 |
| 3 | 3.92 |
| 4 | 7.06 |
| 5 | 8.09 |
| 6 | 2.64 |
| 7 | 2.77 |
| 8 | 2.03 |
| 9 | 17.10 |
| 10 | 0.12 |
| 11 | 8.20 |
| 12 | 9.14 |
| 13 | 5.66 |
| 14 | 9.74 |
| 15 | 8.14 |
| 16 | 7.31 |
| 17 | 12.90 |
| 18 | 6.83 |
| 19 | 6.28 |
| 20 | 11.04 |
| 21 | 1.54 |
| 22 | 9.30 |
| 23 | 5.27 |
| 24 | 2.49 |
| 25 | 6.11 |

As shown in Table 4, the compounds of formula (I) exhibited good 5-lipoxygenase inhibition activity. Therefore, the compounds of formula (I) can be advantageously used for preventing or treating a leukotriene-related disease such as asthma and inflammation diseases in a subject.

### TEST EXAMPLE 2

### In vivo Activity Test

The inventive compound (Example 10) was analyzed for in vivo activities relevant to asthma treatment and the result was compared with those of Zileuton (Zyflo™)(Abbott Laboratories) which is a known 5-lipoxygenase inhibitor, as follows.

(1) Mice Modeling for Sensitization and Airway Challenge

8 Week old female BALB/c mice were obtained from Korean Research Institute of Chemistry Technology, kept in a laminar flow cabinet and divided into 3 groups (5-6 mice per group). The 3 groups of mice were respectively subjected to the following treatments: (1) sham-sensitization plus challenge with phosphate-buffered saline (PBS; ipNeb); (2) sensitization plus challenge with ovalbumin (OVA) (Sigma A5503; Sigma, St. Louis, Mo.) (ipNeb); and (3) sensitization with OVA (ip) plus challenge with OVA (Neb) and a drug (the test compound or Zileuton) (po).

Specifically, the test mice were sensitized with intraperitoneal injection of 20 μg OVA with 4 mg of adjuvant aluminum hydroxide on days 0 and 11. The mice were challenged through the airways with OVA (1% in PBS) on day 11, 21, 22, 23 and 25 after the initial sensitization to induce inflammation. A 50 mg/kg bodyweight dosage of a test drug was orally administered once a day on days 21-25, The mice were assessed 24 hours after the last challenge for the suppressive effect of the drug on the airways of allergic asthma.

(2) Determination of Airway Hyperresponsiveness

Airway hyperresponsiveness (AHR) was determined 24 hours after the final challenge. Each mouse was placed in a barometric plethysmographic chamber and challenged with aerosolized PBS for 3 min, followed by challenging with increasing concentrations of aerosolized methacholine from 0 to 30 mg/ml, at intervals of 5 mg/ml, each for 3 min, and AHR was recorded for 5 minutes thereafter. To evaluate the degree of AHR, the enhanced pause Penh values of the drug sample was obtained during each methacholine challenge, and expressed as a percentage of a basal Penh value obtained in control (PBS) challenge, in which a Penh value was calculated as follows:
Penh=[Tₑ/(RT−1)]×[PEF/PIF]
wherein, Tₑ is expiratory time; RT is relaxation time; PEF is peak expiratory flow; and PIF is peak inspiratory flow.

The results are shown in Table 5.

TABLE 5

| | Methacholine | | | | |
|---|---|---|---|---|---|
| | Concentration | | | | |
| | 0 mg/ml | 5 mg/ml | 10 mg/ml | 20 mg/ml | 30 mg/ml |
| Control | 0.656 | 1.376 | 2.418 | 2.878 | 3.277 |
| (OVA only) | | | | | |
| Zileuton | 0.567 | 1.673 | 2.344 | 2.588 | 2.866 |
| Inventive | 0.383 | 0.756 | 1.339 | 1.863 | 2.724 |
| Compound | | | | | |

As shown in Table 5, the inventive compound exhibited significantly low hyperresponsiveness, compared with Zileuton.

(3) Measurement of IL-4, IL-5 and IL-13 Level

The mice were sacrificeed by pentobarbital overdose (Sigma P3761) 24 hours after AHR measurement and tracheotomy was performed. After ice-cold PBS (0.5 ml) was introduced into the lung, bronchoalveolar lavage fluid (BALF) was obtained by aspiration three times (total 1.5 ml) via tracheal cannulation BALF was centrifuged at 4° C., and the supernatant was collected and stored at −70° C. until use. The amount of cytokines IL-4, IL-5 and IL-13 in BALF was measured by a specific mouse ELISA kit (R&D Systems; Minneapolis, Minn.) and the results are shown in Tables 6 to 8.

TABLE 6
IL-4 Amount (ng/ml) in BALF

| | 1 | 2 | 3 | 4 | 5 | a.v.¹⁾ | s.d.²⁾ |
|---|---|---|---|---|---|---|---|
| Control | 422.03 | 476.73 | 316.4 | 424.01 | 470.2 | 421.872 | 64.1715 |
| (OVA | | | | | | | |
| only) | | | | | | | |
| Zileuton | 274.43 | 321.74 | 358.3 | 266.8 | 204.9 | 285.234 | 58.2696 |
| Inventive | 147.6 | 234.42 | 218.2 | 154.17 | 237.3 | 198.34 | 43.9897 |
| Com- | | | | | | | |
| pound | | | | | | | |

¹⁾Average value
²⁾Standard deviation

TABLE 7
IL-5 Amount (ng/ml) in BALF

| | 1 | 2 | 3 | 4 | 5 | a.v.¹⁾ | s.d.²⁾ |
|---|---|---|---|---|---|---|---|
| Control | 248.82 | 227.21 | 219.52 | 215.05 | 323 | 246.717 | 44.5694 |
| (OVA | | | | | | | |
| only) | | | | | | | |
| Zileuton | 113.31 | 161.01 | 181.44 | 219.27 | 214.3 | 177.864 | 43.3198 |
| In- | 174.05 | 109.83 | 112.81 | 86.353 | 101.6 | 116.923 | 33.5457 |
| ventive | | | | | | | |
| Com- | | | | | | | |
| pound | | | | | | | |

¹⁾Average value
²⁾Standard deviation

TABLE 8
IL-13 Amount (ng/ml) in BALF

| | 1 | 2 | 3 | 4 | 5 | a.v.¹⁾ | s.d.²⁾ |
|---|---|---|---|---|---|---|---|
| Control (OVA only) | 128.45 | 123.16 | 105.09 | 191.87 | 165.9 | 142.89 | 35.1914 |
| Zileuton | 124.37 | 101.3 | 83.84 | 85.45 | 104.3 | 99.842 | 16.4802 |
| Inventive Compound | 75.23 | 48.19 | 41.873 | 80.2 | 76.02 | 64.303 | 17.8335 |

¹⁾Average value
²⁾Standard deviation

As shown in Tables 6 to 8, the inventive compound exhibited the improved inhibiting activity for the formation of cytokines IL-4, IL-5 and IL-13 in BALF, compared with Zileuton.

(4) Histopathology Studies

The lung tissue from each sacrificed mouse was fixed in 10% neutral-buffered formalin for 20 to 24 hours, embedded in paraffin, sliced into 4 μm thickness sections, and stained with H-E solution (hematoxylin, Sigma MHS-16 and eosin, Sigma HT110-1-32). Subsequently, the stained tissue was mounted and cover-slipped with Dako-mounting medium (Dakocytomation; Denmark Carpinteria Calif.). The degree of inflammatory cell infiltration in lung sections, specifically the degree of peri-bronchiole and peri-vascular inflammation was evaluated by a specific standard scale, i.e., scoring with 0-3 (0, no inflammatory cell, 1, few inflammatory populations; 2, a thin ring of inflammatory cells (one to five cell-layer deep); 3, a thick ring of inflammatory cells (more than five cell-layer deep) and averaged. The results are shown in Table 9.

TABLE 9

| Inflammation | Control | | Inventive |
|---|---|---|---|
| Score | (OVA only) | Zileuton | Compound |
| Average value | 2.225 | 1.835 | 1.2427 |
| Standard deviation | 0.224 | 0.195 | 0.232 |

As shown in Table 9, the inventive compound has good suppressive effect on the leukocite infiltration, compared with Zileuton.

While the invention has been described with respect to the above specific embodiments, it should be recognized that various modifications and changes may be made to the invention by those skilled in the art which also fall within the scope of the invention as defined by the appended claims.

What is claimed is:

1. A benzoxazole derivative of formula (I) or a pharmaceutically acceptable salt thereof: 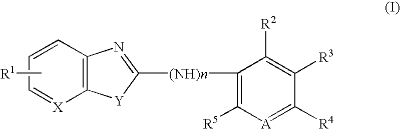
wherein
X is CH or N;
Y is S or O;
n is 0 or 1;
A is CH or N;
R¹ is H, halogen, C₁₋₆ alkyl or C₁₋₆ alkoxy;
R² is H, C₁₋₆ alkyl or halogen-substituted C₁₋₆ mercaptoalkyl;
R³ is H, halogen, C₁₋₆ alkyl, C₁₋₆ haloalkyl or C₁₋₆ alkoxy,
R⁴ is H, halogen, phenylazo, C¹⁻⁶ alkyl, C₁₋₆ mercaptoalkyl or C₁₋₆ alkoxy; and
R⁵ is H or C₁₋₆ alkyl.

2. The benzoxazole derivative of claim 1, wherein
A is CH;
R² is H, or halogen-substituted C₁₋₆ mercaptoalkyl;
R³ is H, halogen or C₁₋₆ haloalkyl;
R⁴ is H, phenylazo, C₁₋₆ alkyl or C₁₋₆ mercaptoalkyl;
R⁵ is H; and
X, Y, n and R¹ are as defined in formula (I).

3. The benzoxazole derivative of claim 1, wherein X is CH, Y is 0, n is 1, R¹ is C₁₋₆ alkyl, R² and R³ are H, R⁴ is C₁₋₆ alkyl, and R⁵ is H.

4. A pharmaceutical composition comprising the benzoxazole derivative of claim 1 or a pharmaceutically acceptable salt thereof as an active ingredient and a pharmaceutically acceptable carrier.

5. A method for inhibiting 5-lipoxygenase in a mammal, which comprises administering the benzoxazole derivative of claim 1 or a pharmaceutically acceptable salt thereof to the mammal.

6. A method for preventing or treating a leukotriene-related disease in a mammal, which comprises administering the benzoxazole derivative of claim 1 or a pharmaceutically acceptable salt thereof to the mammal.

7. The method of claim 6, wherein the leukotriene-related disease is selected from the group consisting of asthma, pertussis, psoriasis, rheumatic arthritis, arthritis, inflammatory bowel disease, cystic fibrosis, acute/chronic bronchitis, sepsis, cardiac myoischemia, cardiac anaphylaxis, ischemia, allergic rhinitis, osteopososis, pain and cancer.
